(19) United States
(12) Patent Application Publication
(10) Pub. No.: US 2005/0272135 A1
(43) Pub. Date: Dec. 8, 2005

(54) Processs for production and purification of fermentation derived organic acids

(75) Inventors: Rathin Datta, Chicago, IL (US); Michael Henry, Batavia, IL (US); Edward J. St. Martin, Libertyville, IL (US)

Correspondence Address: Harry M. Levy,;Emrich and Dithmar, Suite 2080, 125 South Wacker Drive, Chicago, IL, 60606, US

(73) Assignee: The University of Chicago, Chicago, IL (US)

(21) Appl. No.: 10/859,259

(22) Filed: Jun. 2, 2004

(51) Int. Cl. C12P 7/54; C12P 7/52; C07C 51/42

(52) U.S. Cl. 435/140; 435/141; 562/608

(57) ABSTRACT

A method of producing and purifying an organic acid by producing an aqueous solution of the ammonium salt of the organic acid through fermentation and/or bioconversion and neutralization. The solution is heated to thermally crack the ammonium salt of the organic acid producing a vapor phase of ammonia and water and organic acid which is thereafter passed in contact with a membrane permeable to water and ammonia and substantially impermeable to the organic acid vapor to concentrate the aqueous solution of organic acid, and remove the ammonia and excess water.

### CONTRACTUAL ORIGIN OF THE INVENTION

The United States Government has rights in this invention pursuant to Contract No. W-31-109-ENG-38 between the U.S. Department of Energy (DOE) and The University of Chicago representing Argonne National Laboratory.

### FIELD OF THE INVENTION

This invention related to an improved process for the production and purification of fermentation derived organic acids. More specifically this invention relates to an improved method for the recovery and purification of fermentation derived organic acids from their ammonium salts.

### BACKGROUND OF THE INVENTION

Fermentation or bioconversion of many inexpensive and widely available feedstocks to organic acids is well known. These fermentations or bioconversions to produce organic acids operate best at near neutral pH. As the pH drops during fermentation, the metabolism of the organisms and functionality of the key enzymes decreases sharply. This sensitivity to low pH is presently overcome by neutralizing the acids as they are formed with an alkali to produce a salt. Thus, the fermentations do not produce the free acids but rather their salts. Furthermore, the fermentation reactions operate in dilute aqueous media and usually contain many organic and inorganic impurities. Hence, the recovery and purification of organic acids from such streams have to overcome several fundamental separation hurdles. The most important of these is the conversion of the acid salt back to its corresponding acid and alkali. The alkali can then be recycled to neutralize the fermentation/bioconversion process. The other hurdles are removal of impurities and water. For the commercialization of the production of organic acids by fermentation or bioconversion, these separation processes must not only be highly efficient but also economical.

So far, few separation processes have succeeded technically. The electrodialysis (ED) based process of desalting (DSED) and water-splitting (WSED) with bipolar membranes can purify and also neutralize or convert the acid salt back to the corresponding acid and alkali. However, the capital and operating cost and the stringently low divalent ion requirement of the bipolar membranes for WSED step make this process prohibitively expensive for lower value acids. Another approach makes esters directly from ammonium salts of organic acids via a pervaporation assisted esterification process. Organic acids can be produced from these esters at the expense of additional unit operations for the hydrolysis reaction, separation/recycle of the byproduct alcohol, and purification of the acid.

### SUMMARY OF THE INVENTION

It is an object of the present invention to provide an improved process for the production and purification of fermentation or bioconversion derived organic acids.

Another object of the present invention is to provide a method of producing and purifying an organic acid, comprising producing an aqueous solution of the ammonium salt of the organic acid by fermentation and/or bioconversion and neutralization, thermally cracking the ammonium salt of the organic acid to produce a vapor phase of ammonia and water and organic acid, passing the vapor phase in contact with a membrane permeable to water and ammonia and substantially impermeable to the organic acid vapor to concentrate the aqueous solution of organic acid, and removing the ammonia and excess water.

Yet another object of the present invention is to provide a method of type set forth in which the acid is produced by the anaerobic fermentation and an ammonium salt is produced upon neutralization thereof followed by microporous filtration and desalting electrodialysis and evaporation to produce a concentrated ammonium salt of the acid which is then thermally cracked and subjected to pervaporation to separate the acid from ammonia and excess water.

The invention consists of certain novel features and a combination of parts hereinafter fully described, illustrated in the accompanying drawings, and particularly pointed out in the appended claims, it being understood that various changes in the details may be made without departing from the spirit, or sacrificing any of the advantages of the present invention.

### BRIEF DESCRIPTION OF THE DRAWINGS

For the purpose of facilitating an understanding of the invention, there is illustrated in the accompanying drawings a preferred embodiment thereof, from an inspection of which, when considered in connection with the following description, the invention, its construction and operation, and many of its advantages should be readily understood and appreciated.

FIG. 1 is a schematic representation of the process for the production and purification of fermentation derived organic acids; and

FIG. 2 is a schematic of the pervaporation-assisted thermal cracking steps of the process illustrated in FIG. 1.

### DESCRIPTION OF THE PREFERRED EMBODIMENT

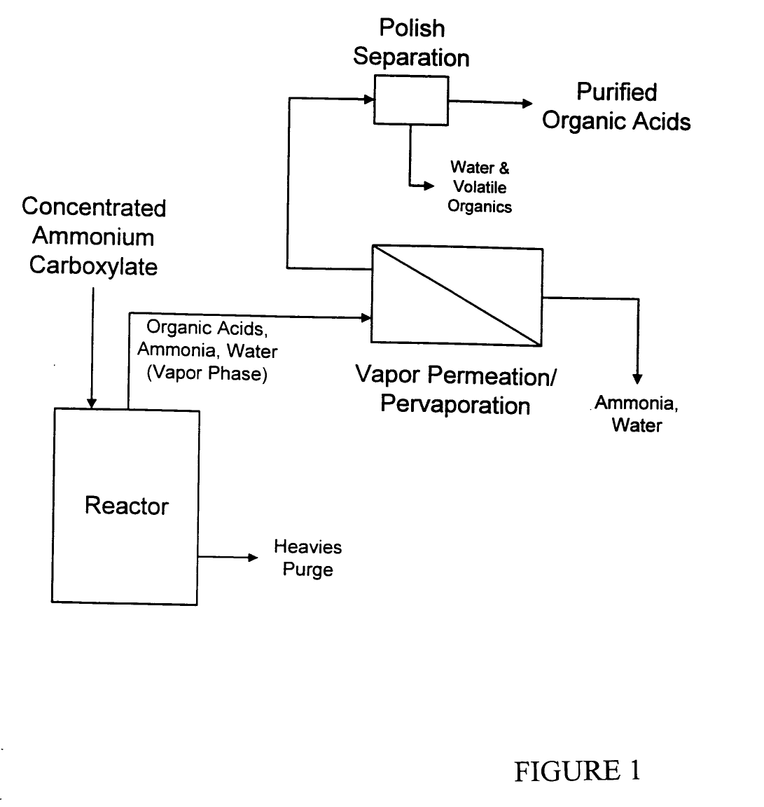

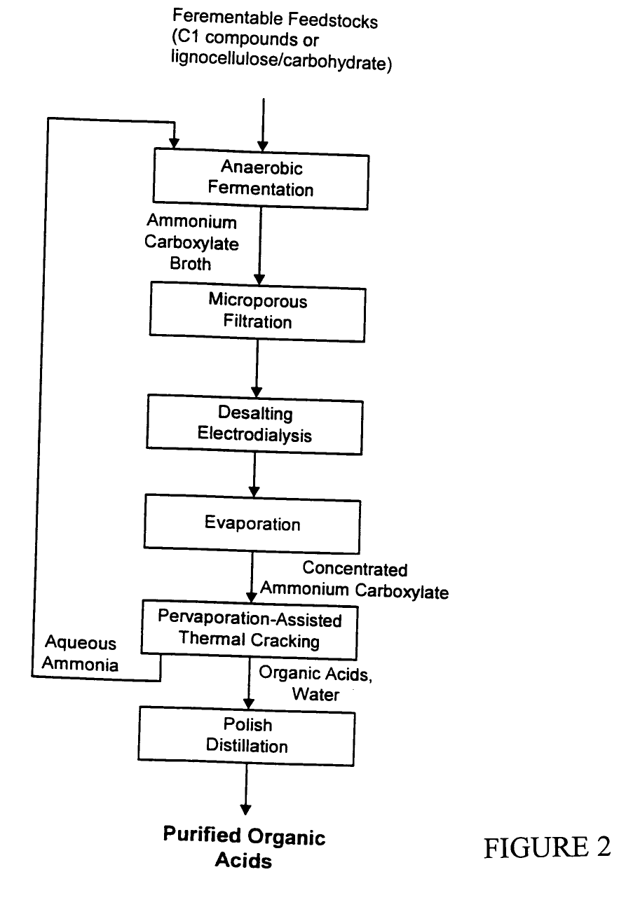

The invention is a novel pervaporation assisted thermal cracking process, which has the potential to overcome the problems enumerated above. Ammonium salts of organic acids are salts of weak acids and base. The acid base bond can be thermally broken at temperatures around 120 to 150° C. For example, ammonium lactate can be thermally cracked between 130 and 150° C. with good kinetics, if the ammonia is rapidly removed. Other ammonium carboxylates have similar cracking properties. Membranes are available, which have a high affinity for water and ammonia, but a low affinity for organics, such as composite multilayer membranes sold by the Sulzer Corporation under designation #2211 or 1211. These are three layer membranes of a modified polyvinyl alcohol top layer, a modified polyacrylonitride middle layer and a stable backing cloth of polyester having thermal stability in the presence of hot (130° C.) vapors of organic acids or solvents. The process of the invention, for the production and purification of fermentation derived organic acids, uses these new membranes. In the process, as shown in FIG. 1, the fermentation/bioconversion broth is neutralized with ammonium hydroxide to produce ammonium carboxylates with high yields and in good concentrations. This broth can also contain microorganism cells and other solids, which can be separated by microporous filtration. The filtered broth is then preferentially subjected to a desalting electrodialysis (DSED) step, which purifies the acid salt from other non-ionic soluble impurities. This partially purified broth is evaporated to a high concentration by energy efficient multi-effect evaporation. This concentrated ammonium carboxylate solution is then fed to the cracker/separator as shown in FIG. 2. There the concentrated solution is heated to cracking temperatures of approximately 120 to 140° C. where the ammonia, water and the acid that is cracked go to the vapor phase. This vapor is circulated past the specialized pervaporation membranes through which readily permeate water and ammonia, thereby separating the ammonia and the water from the organic acid, which does not readily permeate the membranes. Since the membranes are capable of operating at similarly high temperatures (120° to 130° C.), the vapor permeation and ammonia removal are carried out at the same temperature as the thermal cracking. Major advantages of this process include: (1) the acid cannot recombine with the ammonia in the vapor phase to go back to the ammonium salt; and (2) the vaporous acid is separated from the residual heavy impurities that remain in the concentrate.

This process is particularly suitable for volatile organic acids such as formic, acetic, propionic, butyric, isobutyric, etc., which exhibit good ammonium salt cracking characteristics in the temperature range of 120° to 140° C., at which the free acids also boil either at atmospheric or subatmospheric pressures.

The following experimental examples illustrate but do not limit this invention.

### EXAMPLE 1

A simple apparatus was set up to sublimate ammonium acetate solutions at controlled temperatures between 100° C. and 120° C. An HPLC based method was also developed to quantify acetic acid and acetamide concentrations.

Preliminary results from the initial experiments showed:
1a. The rate of sublimation increases with temperature and very good rates can be attained at a temperature of 120° C.
1b. Under these conditions of free sublimation of ammonium acetate solution, the rate of the byproduct acetamide formation is significantly lower than the rate of volatilization. In these experiments the ratio of rates were about 1:50 to 1:100. This means the kinetics are favorable for acetic acid formation and there is not a fundamental kinetic barrier to the development of a high yield separations process.

Further tests conducted at even higher temperatures of 125° C. and 140° C. in aclosed reactor showed that the rate of acetamide formation from an 80% w/w solution of ammonium acetate is very low.

These results are summarized in Tables 1 and 2.

TABLE 1
Summary of Preliminary Ammonium
Acetate Volatilization Kinetics Experimental Data
Open Beaker Tests with 80 wt % Ammonium
Acetate at 120° C. and 30 minutes Reaction Time

| | Acetate | Acetamide | Acetamide to |
|---|---|---|---|
| | Volatilization. | Formation | Acetate |
| | Rate, | Rate, | Mole Ratio, |
| | mol/hr | mol/hr | % |
| Run 1 | 0.368 | 0.008 | 2.3% |
| Run 2 | 0.336 | 0.008 | 2.5% |

TABLE 2
Acetamide to Acetate Mole Ratios in
Closed Reactors at 125 and 140° C.
80 wt % Ammonium Acetate in Water

| | Acetamide to Acetate | |
|---|---|---|
| | Mole Percent Ratio at | |
| Run | Reaction Temperature | |
| Time, min | 125° C. | 140° C. |
| 0 | 0.57% | 1.66% |
| 15 | 0.69% | 3.19% |
| 30 | 1.02% | 4.12% |
| 45 | 1.50% | 5.17% |
| 60 | 1.65% | 6.72% |
| 90 | 2.28% | 8.82% |

### EXAMPLE 2

The Sulzer membranes identified above were tested with liquid phase feed of ammonia, water and ethanol and found that one of the membrane types, Sulzer # 2211, had good water flux, and moderate ammonia flux and the ammonia fluxes increased considerably (˜2.5 fold) with temperature increase from 100° C. to 120° (Table 3).

TABLE 3
Acid-Tolerant Membrane Flux Comparison
All tests conducted with Sulzer Circular, Flat-Sheet
Pervaporation Module in Liquid-Phase Mode

| | | | | | Reactor | |
|---|---|---|---|---|---|---|
| | | | | | Water | |
| | | | | | Conc. | Water |
| Run | Sulzer | Avg. Reactor | Reactor NH₃ | NH₃ | range, | Flux, |
| No. | Membrane | Temp., ° C. | Conc. range, wt % | Flux, kg/m²-hr | wt % | kg/m²-hr |
| 42 | 1201-D | 97 | 2.6-2.4% | ˜0.05 | 8.1-5.5% | ˜0.5 |
| 43 | 1201-D | 117 | 2.6-2.1% | ˜0.15 | 7.4-2.3% | ˜1.3 |
| 50 | 1211-NV | 98 | 2.6-2.4% | ˜0.06 | 8.5-6.3% | ˜0.5 |
| 51 | 1211-NV | 120 | 2.6-2.1% | ˜0.20 | 7.0-2.6% | ˜1.2 |
| 52 | 2211 | 100 | 2.6-2.3% | ˜0.11 | 7.2-4.2% | ˜0.8 |
| 53 | 2211 | 120 | 2.5-1.8% | ˜0.28 | 7.0-1.8% | ˜1.4 |

A vapor permeation module was designed and assembled with #2211 membrane (0.022 m² membrane area) and tested its performance with water, ethanol and ammonia vapor feed and established that this unit could be operated with vapor flow and give fluxes similar to the expected values from the liquid phase tests.

### EXAMPLE 3

For this experiment an 80% (w/w) ammonium acetate solution in water was prepared and heated in a closed reactor to 135° C. and allowed the pressure to build. At the same time the vapor permeation module with the #2211 membrane (0.022 m²) was preheated to ˜120° C. This was necessary to insure that no liquid acetic acid or water would condense on the membrane surface during the test run.

At the beginning of the run the vapor release valve at the top of the reactor was opened and after the vapor passed over the module it was condensed and collected in an enclosed condenser. The permeate from the module was condensed in a cold (0° C.) condenser and any uncondensed permeate vapors were collected in an acid trap (containing ˜25% sulfuric acid) and a cold trap (−50 C). The test run lasted for ˜15 minutes after which no more vapor was being produced by the reactor. Samples from the reactor, condensate, permeate, traps and the vapor were taken and carefully analyzed for free ammonia (by titration), water (by Karl Fischer method) and acetic acid (by HPLC). The masses were also carefully recorded.

The data on compositions, mass balance and flux is summarized in Table 4.

TABLE 4
Ammonium Acetate Cracking & Vapor Permeation Separation
Test Run-2003-4

| | |
|---|---|
| Reactor Temperature, ° C. | 136 |
| Avg. Module Temperature, ° C. | 117 |
| Membrane Sulzer #2211 (m2) | 0.022 |
| Vapor Feed Rate, kg/m²-hr | 70.5 |
| Free Ammonia in Vapor Feed, wt % | 23.5% |
| Water in Vapor Feed, wt % | 43.2% |
| Acetic Acid in Vapor Feed, wt % | 33.4% |
| Ammonia Flux, kg/m²-hr | 0.31 |
| Water Flux, kg/m²-hr | 7.56 |
| Acetic Rejection, % | 99.2% |

The results show:
IIIa. Ammonium acetate vapor containing the three primary components, ammonia, water and acetic acid vapor can be fed to a vapor permeation module with pervaporation membranes at temperatures above the boiling point of acetic acid. This enabled the pervaporation separation to occur in the vapor phase without forming a condensate film on the membrane surface, which would impair the separation because the acetic acid liquid film would react with the ammonia.
IIIb. Under such vapor permeation conditions, water and ammonia are preferentially separated from the acetic acid, which is highly rejected by the membrane.

### EXAMPLE 4

The previous experiments and results with primarily ammonium acetate were conducted at atmospheric or higher than atmospheric pressures, and at or above the boiling point of the acid at these pressures.

However, the process of this invention can be conducted at lower than atmospheric pressure on the vapor feed side. The permeate side is always at a low pressure and temperature and thus there is a chemical potential driving force for the separation.

Aqueous solution of ammonium propionate was used to demonstrate the feasibility. A solution of ammonium propionate was prepared by neutralization of propionic acid with ammonium hydroxide solution, as it would be in a fermentation process. The pH of this was 6.9 and the concentrated solution was ˜70% w/w of ammonium propionate in water. This was fed to an evaporation apparatus heated by a temperature controlled oil bath, and which had a condenser and a vacuum controller. The bath temperature was maintained at 130° C., which would be the typical operating temperature of the vapor permeation membrane separator. The vacuum was provided by a water flow aspirator and controlled by a control valve that aspirated atmospheric air. The condenser was maintained at ˜0° C., which would be typical permeate side temperature. Approximately 200 g of the concentrated ammonium propionate solution was charged to the evaporator and the vaporization was run for 60 minutes at an average bath temperature of 130° C., pressure of 500 millibars (˜400 mm Hg vacuum), and a condenser temperature of ° C. Weights and samples of the feed, condensate and residual feed concentrate were measured and analyzed. An HPLC based method was used to quantify propionic acid and propionamide concentrations and a Karl Fischer apparatus was used to measure water content. The collected condensate weight was approximately 80 g and apart from water and ammonia, it contained 18% w/w propionic acid. The residual feed had very little water (˜1%) and propionamide (˜3%) and was primarily ammonium propionate/propionic acid.

These results show:

IV a. Ammonium propionate can be thermally cracked and volatilized at 130° C. which is lower than the atmospheric boiling point of propionic acid (141° C.)
IV b. The propionamide (undesirable byproduct) formation rate is relatively low.
IVc. This volatilization at sub-atmospheric pressures provides ammonia, propionic acid and water in the vapor phase and the ammonia and water would be separated from the acid under the typical operating conditions of the vapor permeation process.

This also shows that the inventive process is suitable for many fermentation derived ammonium salts of volatile organic acids such as formic, acetic, butyric, isobutyric and 3-hydroxy propionic. A table with atmospheric and sub-atmospheric boiling points of these acids is provided below.

| Organic | Boiling Point at | Boiling Point at |
|---|---|---|
| Acid | 760 mm Hg, ° C. | 400 mm Hg, ° C. |
| Formic | 100.8 | 80.4 |
| Acetic | 118.0 | 98.4 |
| Propionic | 141.1 | 121.4 |
| I-Butyric | 154.0 | 133.7 |
| N-Butyric | 163.3 | 145.9 |
| 3-hydroxy | 162.0 | — |
| propionic | | |

While there has been disclosed what is considered to be the preferred embodiment of the present invention, it is understood that various changes in the details may be made without departing from the spirit, or sacrificing any of the advantages of the present invention.

1. A method of producing and purifying an organic acid, comprising producing an aqueous solution of the ammonium salt of the organic acid by fermentation and/or bioconversion and neutralization, thermally cracking the ammonium salt of the organic acid to produce a vapor phase of ammonia and water and organic acid, passing the vapor phase in contact with a membrane permeable to water and ammonia and substantially impermeable to the organic acid vapor to concentrate the aqueous solution of organic acid, and removing the ammonia and excess water.

2. The method of claim 1, wherein the boiling point of the organic acid at atmospheric pressure is less than about 200° C.

3. The method of claim 1, wherein the organic acid is one or more of acetic, formic, propionic, 3-hydroxy propionic, butyric or iso-butyric.

4. The method of claim 1, wherein the aqueous solution containing ammonium salts is filtered to remove microorganisms cells and solids.

5. The method of claim 1, wherein the aqueous solution of ammonium salts is subjected to electrodialysis to remove non-ionic impurities.

6. The method of claim 1, wherein the membrane has multiple layers containing a polyvinyl alcohol polymer layer and a polyacrylonitrile layer having chemical and physical stability in hot organic acids.

7. The method of claim 1, wherein the membrane is contacted with the vapor phase feed at less than atmospheric pressure.
